(19) United States
(12) Patent Application Publication
(10) Pub. No.: US 2022/0389365 A1
(43) Pub. Date: Dec. 8, 2022

(54) ENZYME COMPOSITION AND METHOD OF USING THE SAME FOR INDUSTRIAL CLEANING IN BIOMASS-BASED PROCESSING

(71) Applicant: Larry Camden Fowler, II, Ames, IA (US)

(72) Inventor: Larry Camden Fowler, II, Ames, IA (US)

(21) Appl. No.: 17/831,112

(22) Filed: Jun. 2, 2022

Related U.S. Application Data
(60) Provisional application No. 63/196,592, filed on Jun. 3, 2021.

(51) Int. Cl. C12M 1/00; B01D 1/30; C11D 3/386; C11D 11/00; C12N 9/28

(52) U.S. Cl. CPC C12M 39/00; B01D 1/30; C11D 3/38609; C11D 11/0041; C11D 11/0064; C12N 9/2417

(57) ABSTRACT

Disclosed herein are embodiments of an enzyme composition for use in cleaning components of biomass-based processing. In some embodiments, the enzyme composition comprises a surfactant or is used in combination with a surfactant-containing composition. The enzyme composition can be administered to a process stream or directly to a process screen used in biomass-based processing to facilitate removing starch build-up on the process screen.

### CROSS REFERENCE TO RELATED APPLICATION

This application claims the benefit of and priority to the earlier filing date of U.S. Provisional Patent Application No. 63/196,592, filed on Jun. 3, 2021, the entirety of which is incorporated herein by reference.

### FIELD

The present disclosure is directed to enzyme composition embodiments and method embodiments wherein the enzyme composition is used to clean equipment used in biomass-based processing.

### BACKGROUND

Biomass-based processing methods, such as grain processing (e.g. corn-to-ethanol production), utilize materials that can result in clogging and dirtying the equipment used in such methods. For example, solids can build up and contaminated equipment surfaces. It therefore becomes necessary to clean the equipment; however, conventional methods for doing so often require shutting down an industrial plant to facilitate cleaning, which results in delays in product formation and increases costs associated with the process. Cleaning-in-place (CIP) is an example of a method whereby a plant typically must be shut down to conduct the cleaning, despite the fact that major disassembly is typically not required. CIP processes can be used to clean interior surfaces and internal components of the equipment used in biomass-based processing; however, the need to stop the process for a period of time (e.g., 10-12 hours) to conduct the cleaning results inefficiency and higher costs for a plant. There exists a need in the art for compositions and methods that can facilitate industrial equipment cleaning used in biomass-based processing and that are more cost effective and environmentally-friendly than using conventional cleaners/cleaning methods.

### SUMMARY

Disclosed herein are embodiments of a method for cleaning a process screen. In some embodiments, the method comprises administering an enzyme composition comprising an amylase enzyme, a protease enzyme, a lipase enzyme, or any combination thereof to a process stream produced during biomass-based processing, wherein the process stream is a process stream that is exposed to fiber separation or protein separation; provided that the enzyme composition is not administered directly to a fermentation unit or a liquefaction unit of the biomass-based processing.

In some other embodiments, the method comprises: administering an enzyme composition comprising an amylase enzyme, a protease enzyme, a lipase enzyme, or any combination thereof to a process stream produced during biomass-based processing, wherein (i) the process stream flows between a liquefaction unit and a fermentation unit used in the biomass-based processing; (ii) the process screen is located between the liquefaction unit and the fermentation unit; and (ii) the enzyme composition is administered upstream of the process screen; provided that the enzyme composition is not administered directly to the fermentation unit or the liquefaction unit of the biomass.

In yet other embodiments, the method comprises administering an enzyme composition comprising an amylase enzyme, a protease enzyme, a lipase enzyme, or any combination thereof to a process stream produced during biomass-based processing, wherein (i) the process stream flows from a fermentation unit to a protein separation unit configured to separate protein from whole stillage; and (ii) the enzyme composition is administered upstream of the process screen; provided that the enzyme composition is not administered directly to the fermentation unit.

In yet other embodiments, the method comprises administering an enzyme composition comprising an amylase enzyme to the process screen during biomass-based processing, wherein (i) the process screen is located between the liquefaction unit and the fermentation unit; and (ii) the enzyme composition is administered to the process screen by way of a cleaning stream that is separate from any process stream of the biomass-based processing.

The foregoing and other objects and features of the present disclosure will become more apparent from the following detailed description, which proceeds with reference to the accompanying figures.

### BRIEF DESCRIPTION OF THE DRAWINGS

FIG. 1 is a block diagram of certain components and/or steps involved in an exemplary biomass-based processing method, such as that conducted in an ethanol and oil processing plant.

FIG. 2 is a block diagram of certain components and/or steps involved in an exemplary biomass-based processing plant, such as a plant where a protein separation process is used to separate protein separated from whole stillage produced in a corn dry-milling process for making ethanol.

### DETAILED DESCRIPTION

### I. Overview of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Administering: An affirmative action whereby an enzyme composition is added to a process stream from an enzyme composition containment unit. Adding the enzyme composition can also be referred to herein as “dosing.”

Cleaning-in-place (or CIP): A method of cleaning the interior surfaces of a system or system components used in industrial processing without major disassembly.

Downstream: As used herein, the term “downstream” refers to a point in a process stream after a liquefaction unit where an enzyme composition is administered. Immediately downstream means a downstream point in the process stream that is proximate to the administration site of the enzyme composition.

Enzyme Composition Containment Unit: A unit that contains an enzyme composition according to the present disclosure and that is not a physical component used to convert a feedstock to one or more intermediate products and/or one or more final products in a biomass-based processing method. The enzyme composition containment unit is not located directly in the processing stream and instead is only fluidly coupled to the processing stream in a manner such that the enzyme composition can be delivered to the processing stream or a component of the processing stream from the enzyme composition containment unit. Components of the processing stream (e.g., feedstocks, intermediate products, and/or final products) do not enter the enzyme composition containment unit.

Enzyme: A protein molecule that is capable of catalyzing a chemical reaction. For example, an amylase is an enzyme that catalyzes starch hydrolysis. Exemplary enzymes of the present disclosure are described herein.

Pulse Dosing: Intermittent dosing, wherein periods of dosing are alternated with periods of no dosing.

Scale: A hard mineral coating or corrosion deposit composted of solids and sediments that collect on components used in biomass-based processing as a process stream flows through or over the components.

Surfactant: A compound that reduces surface tension when dissolved in water or water solutions, or that reduces interfacial tension between two liquids. A surfactant molecule typically has a polar or ionic “head” and a nonpolar hydrocarbon “tail.” Upon dissolution in water, the surfactant molecules aggregate and form micelles, in which the nonpolar tails are oriented inward and the polar or ionic heads are oriented outward toward the aqueous environment. Micelles typically are spherical in shape and small, with diameters of less than about 10 nm. The nonpolar tails create a nonpolar “pocket” within the micelle.

Upstream: When referring to the flow of a process stream into a process screen, the term “upstream” refers to a point anywhere in the process stream prior to the process screen but after any liquefaction unit.

### II. Introduction

Industrial-scale biomass-based processing often experiences slow-down and/or inefficiency due to the presence/production of solid contaminants that become deposited on surfaces and other structural features of the equipment used in processing. For example, many solid deposits resulting from biomass-based processing can be particularly difficult to remove, especially if the soil is thermally degraded because the material has been heated during processing. Removing these solid contaminants is imperative to maintain efficiency of the industrial process; however, long cleaning times are needed with conventional cleaning methods, even CIP methods. CIP methods can take several hours to complete, which results in having to shut down the plant for extended periods of time since the processing and CIP method cannot be conducted simultaneously.

Disclosed herein are enzyme composition embodiments for use in biomass-based processing that facilitate cleaning equipment used in the process so as to remove or substantially decrease the amount of solid build-up that can be produced during processing. Also disclosed are method embodiments of using the enzyme composition to prevent or at least substantially reduce solid build-up on equipment used in the process. The enzyme composition and method embodiments of the present disclosure facilitate the ability to run the biomass-based process without significant interruptions in the process that otherwise would be needed for CIP processes. The disclosed enzyme composition and method embodiments are environmentally-friendly and can be utilized with established infrastructure. In particular embodiments, the enzyme composition and method embodiments of the present disclosure facilitate higher yields of the products (e.g., ethanol, protein, or other products obtained from biomass) produced during the biomass-based processing.

In some of the disclosed embodiments, biomass-based processing can be carried out in different types of processing plants, such as a grain processing plant or an ethanol processing plant. The ethanol may be ethanol from corn, milo, or wheat. Grains can include, but are not limited to, corn, corn mash, rye, wheat, barley, or combinations thereof. The process stream of such processing plants may be a process stream comprising (i) a grain, grain-derived products (e.g., stillage, protein, or the like), or a combination thereof, or (ii) a process condensate, a rinse fluid, a cleaning fluid, or any combination thereof. In some embodiments, the process stream includes, but is not limited to, mash, whole stillage, and thin stillage. In some embodiments, the process stream comprises a process condensate.

Processing facilities used for biomass-based processing can comprise one or more structural components selected from a milling unit, a heating/cooking and liquefaction unit, a heat exchanger unit (also referred to as a mash bank), a propagation unit (e.g., for propagating yeast), a fermentation unit, a distillation unit, an evaporation unit, a centrifuge unit, a fiber separation unit, a protein separation unit, an oil separation unit, or any combination thereof. Each structural component or unit may comprise one or more components arranged in series or in parallel. For instance, a distillation unit may comprise one, two, three, or more distillation columns. An evaporation unit may comprise, one, two, three, or more evaporators. Fiber separation and/or protein units independently may comprise a plurality of separation screens; for instance, the fiber separation unit may sequentially comprise primary, secondary, and tertiary separation screens.

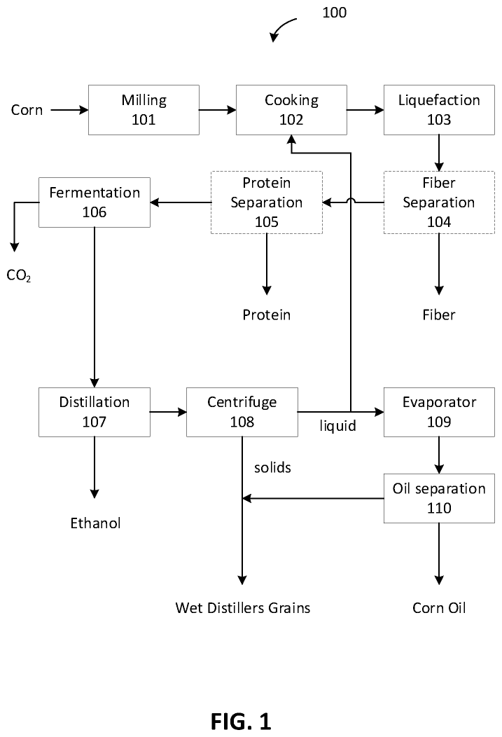

A block diagram of an exemplary corn-to-ethanol processing facility 100 is shown in FIG. 1, which shows certain structural components and/or steps involved in corn-to-ethanol processing. Although the diagram of FIG. 1 shows one exemplary arrangement of the facility components, a person of ordinary skill in the art will understand that other arrangements are also possible with the benefit of the present disclosure and the exemplary arrangement of FIG. 1 is not necessarily preferred. The processing facility 100 includes a milling unit 101 where corn is milled to form a corn mash or process stream. The milled corn process stream flows through a cooking unit 102 and a liquefaction unit 103, in which the corn mash is cooked and degraded into its component parts (e.g., fiber, protein, oils, etc.). In some processing facilities, the process stream then flows through a fiber separation unit 104 and a protein separation unit, which remove fiber and protein, respectively, from the process stream. In other processing facilities, the process stream flows directly from the liquefaction unit 103 to the fermentation unit 106. Although the exemplary arrangement of FIG. 1 shows fiber separation prior to protein separation, a person of ordinary skill in the art understands, particularly with the benefit of the present disclosure, that protein separation can be performed prior to fiber separation if desired or either process can be performed on its own. The process stream then flows into the fermentation unit 106 where the process stream is fermented to produce ethanol. The fermented process stream flows into the distillation unit 107, which may comprise one or more distillation columns. Ethanol is distilled from the process stream, and the remainder of the stream flows into the centrifuge 108, wherein solids are separated from the process stream. The process stream is then split, with part of the stream being recycled back to cooking unit 102 and part of the stream flowing to an evaporator unit 109, wherein low molecular-weight components (e.g. water and other volatile components) are removed from the process stream, providing a fluid stream comprising a syrup. The syrup then flows into the oil separation unit 110, which separates corn oil from any remaining solids that formed in the evaporator unit 109. Although not shown explicitly, the person of ordinary skill in the art understands, particularly with the benefit of the present disclosure, that the cooking unit 102, liquefaction unit 103, fermentation unit 106, distillation unit 107, and/or evaporator unit 109 may comprise one or more heat exchangers.

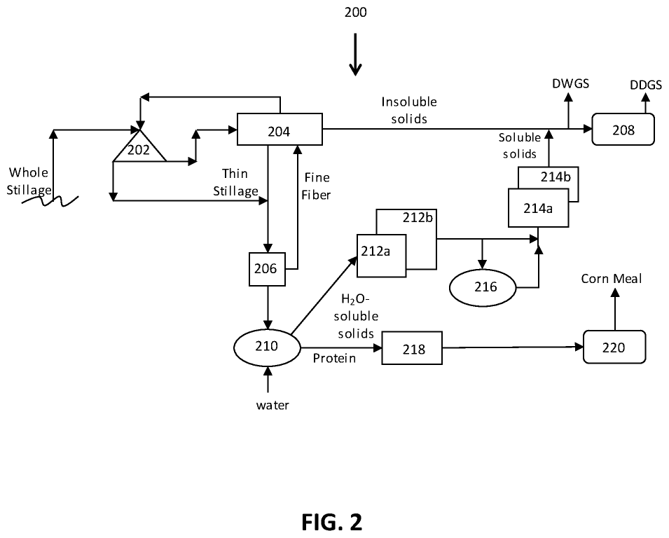

In some embodiments, the processing facility can include components used for separating protein from whole stillage produced in biomass-based processing (e.g., corn dry-milling processes and the like). FIG. 2 schematically illustrates a system embodiment of a biomass-based processing embodiment, which involves protein separation from whole stillage produced in a corn dry-milling process. The whole stillage that undergoes protein separation typically comprises a slurry of soluble and insoluble solids (e.g., spent grains from any distillation and dehydration steps used in the dry-milling). The insoluble solids can include protein, fiber, oil, and sugars and the like. As shown by FIG. 2, whole stillage can be transferred to unit 200 from a component of the corn dry-milling process and passed through screen 202. Screen 202 is positioned upstream of filtration centrifuge 204, so as to facilitate separating insoluble solids portion from the thin stillage portion by initially filtering out desirable amounts of water and protein and, in some embodiments, small fiber fines from the whole stillage. Enzyme composition embodiments disclosed herein can be administered to screen 202. Thin stillage underflow from filtration centrifuge 204 can be transferred to join up with the underflow from screen 202. Then, the thin stillage can be passed through second screen 206 to further separate any fine fiber from the thin stillage. Insoluble solids from filtration centrifuge 204 can be passed to dryer 208, whereas the remaining thin stillage can pass to centrifuge 210 to separate water-soluble solids from protein. Water-soluble solids can be passed to a series of evaporators 212a and 212b and 214a and 214b (with an optional passage through an oil recovery centrifuge 216). Any number of evaporators can be used. The separated protein can be passed from centrifuge 210 to decanter centrifuge 218 and then to dryer 220.

### III. Enzyme Composition Embodiments

Enzyme composition embodiments of the present disclosure comprise an enzyme. In particular embodiments, the enzyme can be an enzyme that facilitates hydrolyzing starch and/or other sugar-based solid components into simple sugars. In particular embodiments, the enzyme is an amylase, which can be a natural or a genetically engineered amylase, or a combination thereof; a protease, which can be a natural or a genetically engineered protease, or a combination thereof; a lipase, which can be a natural or a genetically engineered lipase, or a combination thereof; or any combination of such enzymes. Exemplary amylases include, but are not limited to, α-amylase, β-amylase, γ-amylase, or combinations thereof. In some embodiments, the α-amylase is that of a genetically modified Bacillus stearothermophilus strain, or a Bacillus licheniformis strain, or a combination thereof. The enzyme of the enzyme composition disclosed herein is a component that is separate from any enzyme used in any fermentation, liquefaction, and/or saccharification processing step of a biomass-based processing method (e.g., grain processing method or ethanol processing method) to break down a feedstock into hydrolyzed components. As such, in particular embodiments, the enzyme composition is not administered to a fermentation broth or a liquefaction fraction for this purpose. The enzyme may be the same, chemically, as any enzyme used in fermentation, liquefaction, or saccharification, but it used in addition to any such enzyme and thus can make-up a separate component administered to a process stream that is distinct from any enzyme used for fermentation, liquefaction, or saccharification in the biomass-based processing method.

In some embodiments, the enzyme composition can further comprise additional components, such as water or an organic solvent. In particular embodiments, the enzyme composition may not comprise a surfactant but instead is used in conjunction with a surfactant-containing composition. Suitable surfactant-containing compositions can include, but are not limited to, surfactant-based detergent cleaning formulations (e.g., PHIBROCLEAN™), cleaning formulations comprising nitric acid and a detergent (e.g., PHIBRO AC™), scale inhibitor formulations (e.g., PHIBRO SI™), organic deposit control formulations (e.g., PHIBRO DC™), or any combination thereof. In such embodiments, the enzyme composition and the surfactant-containing composition can be combined prior to administering the compositions to a process stream or they can be administered to the process stream separately and sequentially in any order. In an independent embodiment, the enzyme composition comprises a combination of the enzyme and a surfactant. In any enzyme composition embodiments comprising a surfactant, or any enzyme composition embodiments that are used in conjunction with a surfactant-containing composition, the surfactant can be an anionic or a non-ionic surfactant. In particular embodiments, the surfactant is non-ionic. In some additional embodiments, the surfactant is a biodegradable surfactant with low foaming action and low toxicity. In some embodiments, the enzyme composition can be administered in conjunction with one or more different types of a surfactant-containing composition. In some embodiments, the surfactant can be selected from a polysorbate surfactant, such as polyoxyethylene (20) sorbitan monolaurate, polyoxyethylene (20) sorbitan monopalmitate, polyoxyethylene (20) sorbitan monostearate, or polyoxyethylene (20) sorbitan monooleate; a fatty acid ethoxylate, such as ethoxylated versions of lauric acid, oleic acid, stearic acid, palmitic acid. Neither the enzyme composition nor the surfactant-containing composition comprises a silica component, such as silica particles (e.g., silica nanoparticles or silica microparticles). In yet additional embodiments, the enzyme composition does not comprise a buffer, such as a phosphate buffer.

The amount of enzyme included in the enzyme composition can range from 15 ppm to 500 ppm, such as 50 ppm to 500 ppm, or 100 ppm to 500 ppm, or 150 ppm to 500 ppm. In some other embodiments, the enzyme composition can be a neat composition consisting of the enzyme or the enzyme and solvent (e.g., water or organic solvent).

### IV. Method Embodiments

Disclosed herein are embodiments of a method comprising administering an enzyme composition as described herein to a processing stream of a biomass-based process. In contrast to enzymes that are added to facilitate fermentation, liquefaction, and/or saccharification of a biomass feedstock into different products (e.g., oil or ethanol or other alcohols), the enzyme composition embodiments disclosed herein are used to remove starch from surfaces of processing components used within a system for biomass-based processing. As such, the enzyme compositions are utilized to clean such components. For example, the enzyme composition can be used to remove starch from the surface of a process screen component used in biomass-based processing, such as a separation screen used for fiber separation and/or protein separation. The starch can be wet starch, dry starch, or a combination thereof. The enzyme composition can be used to clean physical components using in biomass processing, such as pipes, discs, filters, screens (e.g., paddle screens, pressure screens, filtration screens, etc.), and the like.

In particular embodiments, the method comprises administering the enzyme composition to the processing stream at, or upstream of, a process screen used in fiber separation. In some embodiments, the method comprises administering the enzyme composition to the processing stream at, or upstream of, a process screen used in protein separation. In other embodiments, the method comprises administering the enzyme composition directly to the process screen using a cleaning stream that is separate from the processing stream. In yet additional embodiments, the enzyme composition can be administered to the processing stream and a separate cleaning stream simultaneously or sequentially. In yet additional embodiments, the enzyme composition can be used in a CIP process whereby processing is stopped and the processing screen is removed from the system and then cleaned with the enzyme composition. In any such embodiments, the method can further comprise administering a surfactant-containing composition to the same processing and/or cleaning stream as the enzyme composition simultaneously or sequentially with the enzyme composition. In particular embodiments, the process screen is located downstream of the liquefaction unit and upstream of the fermentation unit. In embodiments involving protein separation, the process screen is located downstream of the liquefaction unit and the fermentation unit and further is positioned upstream of a centrifuge.

In some embodiments, the process screen is part of a fiber separation unit. The fiber separation unit may further comprise one or more additional screens that are arranged in sequence. In some such embodiments, the method can comprise administering the enzyme composition upstream of all screens contained in the fiber separation unit. In yet other embodiments, the method can comprise administering the enzyme composition upstream of each screen contained in the fiber separation unit using independent dosing steps wherein one portion of the enzyme composition is administered upstream of a first screen, a second portion of the enzyme composition is administered upstream of a second screen, a third portion of the enzyme composition is administered upstream of a third screen, and so on. In an independent embodiment, the enzyme composition is not administered to the liquefaction unit or the fermentation unit.

In some embodiments, the process screen is part of a protein separation unit. The protein separation unit may further comprise one or more additional screens that are arranged in sequence. In some such embodiments, the method can comprise administering the enzyme composition upstream of all screens contained in the protein separation unit. In yet other embodiments, the method can comprise administering the enzyme composition upstream of each screen contained in the protein separation unit using independent dosing steps wherein one portion of the enzyme composition is administered upstream of a first screen, a second portion of the enzyme composition is administered upstream of a second screen, a third portion of the enzyme composition is administered upstream of a third screen, and so on.

In embodiments wherein the enzyme composition comprises a surfactant or is used in conjunction with a surfactant-containing composition, the enzyme and the surfactant can exhibit a synergistic effect in degrading the starch and thus facilitating its removal from the physical components of the processing system. In such embodiments, more starch can be degraded and removed from components of the biomass-based processing method than is degraded by the enzyme or the surfactant individually. In exemplary embodiments, the effect of the surfactant-containing composition and the enzyme composition are more than additive. In particular embodiments, the method embodiments (and composition used therein) disclosed herein will improve flow through components of the process, such as through one or more process screens. In such embodiments, flow improvement is indicated by flow rates wherein flow of liquid through the screen compared to incoming total flow are the same or substantially similar (that is, within less than 50%, such as less than 25%, or less than 25%, or less than 10%, or less than 5% of one another).

In some embodiments, the enzyme composition can be administered to the process stream in an amount selected to provide a desired concentration of enzyme in the process stream. In some such embodiments, the enzyme composition can be administered in an amount ranging from 15 ppm to 500 ppm, such as 15 ppm to 150 ppm, or 150 ppm to 500 ppm. In particular embodiments, the enzyme composition is administered in an amount of 500 ppm.

In some embodiments, the enzyme composition can be administered using pulse dosing. In such embodiments, pulse dosing may comprise administering the enzyme composition into the process stream for a period of x seconds every y minutes, with no administration of the enzyme composition between the periods of x seconds. In some embodiments, x and y independently are from 1 to 500, such as 1-250, 1-150, 5-150, 5-100, 10-100, 10-90, 15-90, or 15-60. In certain examples, x and y independently are 5, 10, 15, 30, 45, 60, or 90. In some non-limiting examples, the enzyme composition is pulse dosed into the process stream for a period of 5 seconds every 5 minutes, 10 seconds every 10 minutes, 15 seconds every 15 minutes, 30 seconds every 30 minutes, 45 seconds every 45 minutes, 60 seconds every 60 minutes, or 90 seconds every 90 minutes. In any of the foregoing or following embodiments, x and y may be the same or different. In some embodiments, x and y are the same, e.g., 30 seconds every 30 minutes. In some embodiments, x and y are different, e.g., 15 seconds every 30 minutes or 45 seconds every 30 minutes. In such embodiments, the surfactant can be similarly dosed.

Administering the cleaning formulation into the process stream via pulse dosing may be performed by any suitable method. In some embodiments, administering is performed by flowing, injecting, or spraying the enzyme composition into the process stream or any separate cleaning stream. Administration may be performed at a continuous or substantially continuous rate throughout each period of x seconds. In any pulse dosing embodiments, the enzyme composition may be pulse dosed into the process stream or any separate cleaning stream to provide any desired or effective concentration of the enzyme composition in the process stream or any separate cleaning stream during the period of x seconds. In some embodiments, the enzyme composition is pulse dosed into the process stream to provide a concentration of 15 ppm to 1500 ppm, such as 50 ppm to 1500 ppm, or 100 ppm to 1500 ppm, or 150 ppm to 1500 ppm, or 500 ppm to 1500 ppm of the enzyme composition in the process stream or any separate cleaning stream during the period of x seconds. The concentration may be determined at or immediately downstream of the site where the enzyme composition is administered. In particular embodiments, the concentration is 500 ppm, 1,000 ppm, or 1500 ppm.

### V. Overview of Several Embodiments

Disclosed herein are embodiments of a method for cleaning a process screen, comprising: administering an enzyme composition comprising an amylase enzyme, a protease enzyme, a lipase enzyme, or any combination thereof to a process stream produced during biomass-based processing, wherein the process stream is a process stream that is exposed to fiber separation or protein separation; provided that the enzyme composition is not administered directly to a fermentation unit or a liquefaction unit of the biomass-based processing.

In any or all of these embodiments, the method is a method for cleaning a process screen, comprising: administering an enzyme composition comprising an amylase enzyme, a protease enzyme, a lipase enzyme, or any combination thereof to a process stream produced during biomass-based processing, wherein (i) the process stream flows between a liquefaction unit and a fermentation unit used in the biomass-based processing; (ii) the process screen is located between the liquefaction unit and the fermentation unit; and (ii) the enzyme composition is administered upstream of the process screen; provided that the enzyme composition is not administered directly to the fermentation unit or the liquefaction unit of the biomass.

In any or all of these embodiments, the method is a method for cleaning a process screen, comprising: administering an enzyme composition comprising an amylase enzyme, a protease enzyme, a lipase enzyme, or any combination thereof to a process stream produced during biomass-based processing, wherein (i) the process stream flows from a fermentation unit to a protein separation unit configured to separate protein from whole stillage; and (ii) the enzyme composition is administered upstream of the process screen; provided that the enzyme composition is not administered directly to the fermentation unit.

In any or all of the above embodiments, administering comprises administering the enzyme composition from an enzyme composition containment unit to the process stream, wherein the enzyme composition containment unit is fluidly coupled to the process stream.

In any or all of the above embodiments, administering comprises pulse dosing the enzyme composition into the process stream for a period of x seconds every y minutes with no administration of the enzyme composition between the periods of x seconds, wherein x and y independently range from 1 to 500.

In any or all of the above embodiments, x and y independently are from 15 to 90.

In any or all of the above embodiments, x=y=30.

In any or all of the above embodiments, an amount of the enzyme composition is administered into the process stream to provide a concentration of 15 ppm to 1,500 ppm of the enzyme composition in the process stream during the period of x seconds.

In any or all of the above embodiments, the amylase enzyme is an α-amylase, a β-amylase, a γ-amylase, or any combination thereof.

In any or all of the above embodiments, the enzyme composition is a separate composition from any composition comprising an enzyme that is used to in any liquefaction, fermentation, and/or saccharification step of the biomass-based processing.

In any or all of the above embodiments, the enzyme composition further comprises a surfactant.

In any or all of the above embodiments, the method further comprises combining the enzyme composition with a surfactant-containing composition.

In any or all of the above embodiments, combining the enzyme composition with the surfactant-containing composition comprises mixing the enzyme composition with the surfactant-containing composition prior to administering the enzyme composition into the process stream or separately, or sequentially or simultaneously adding the enzyme composition and the surfactant-containing composition to the process stream.

In any or all of the above embodiments, the method further comprises administering the enzyme composition to one or more process screens used in a fiber separation unit of the biomass-based processing.

Also disclosed herein are embodiments of a method for cleaning a process screen, comprising: administering an enzyme composition comprising an amylase enzyme, a protease enzyme, a lipase enzyme, or any combination thereof to the process screen during biomass-based processing, wherein (i) the process screen is located between the liquefaction unit and the fermentation unit; and (ii) the enzyme composition is administered to the process screen by way of a cleaning stream that is separate from any process stream of the biomass-based processing.

In any or all of the above embodiments, administering comprises administering the enzyme composition from an enzyme composition containment unit to the cleaning stream, wherein the enzyme composition containment unit is fluidly coupled to the cleaning stream.

In any or all of the above embodiments, administering comprises pulse dosing the enzyme composition into the cleaning stream for a period of x seconds every y minutes with no administration of the enzyme composition between the periods of x seconds, wherein x and y independently range from 1 to 500.

In any or all of the above embodiments, x and y independently are from 15 to 90.

In any or all of the above embodiments, x=y=30.

In any or all of the above embodiments, an amount of the enzyme composition is administered into the cleaning stream to provide a concentration of 15 ppm to 1,500 ppm of the enzyme composition in the cleaning stream during the period of x seconds.

In any or all of the above embodiments, the amylase enzyme is an α-amylase, a β-amylase, a γ-amylase, or any combination thereof.

In any or all of the above embodiments, the enzyme composition further comprises a surfactant.

In any or all of the above embodiments, the method further comprises combining the enzyme composition with a surfactant-containing composition.

### VI. Examples

### Example 1

In this example, an enzyme composition according to the present disclosure is administered to a fluid process stream (corn mash) of an ethanol- and oil-processing plant upstream of a process screen located between the liquefaction unit and the fermentation unit. The enzyme composition is administered to the process stream to provide a concentration of 500 ppm. Following administration of the enzyme composition, deposited material on the process screen is removed easily, such as without hydroblasting and instead using standard washing. Effects on flow rate through the process screen without the enzyme composition and with the enzyme composition being administered are evaluated. Results are analyzed using a one-way Anova test and student's t test. Enzyme composition administration improves the flow rate consistency. For example, much less variability in flow rates and an increased interval between standard screen cleanings can be observed as enzyme composition administration improves the flow rate consistency, largely eliminates low flow rate occurrences, and maintains stable flow ratios without flow rate decline between screen washes.

### Example 2

In this example, an enzyme composition according to the present disclosure is administered to a fluid process stream (corn mash) of an ethanol- and oil-processing plant upstream of a process screen located between the liquefaction unit and the fermentation unit, along with a surfactant-containing composition. The compositions are administered separately such that the enzyme composition is administered to the process stream. The enzyme composition and the surfactant-containing composition work synergistically to remove deposited material on the process screen, wherein the level of starch removed from the process screen is more than an additive amount observed when using just the surfactant-containing composition and enzyme composition alone. Effects on flow rate through the process screen without the enzyme and surfactant-containing compositions and with the enzyme and surfactant compositions being administered are evaluated. Results are analyzed using a one-way Anova test and student's t test. The two compositions synergistically improve the flow rate consistency. For example, much less variability in flow rates and an increased interval between standard screen cleanings can be observed as administering the dual compositions improves the flow rate consistency, largely eliminates low flow rate occurrences, and maintains stable flow ratios without flow rate decline between screen washes.

### Example 3

An enzyme composition is administered into a fluid process stream of a processing plant. The processing plant may be an ethanol processing plant, a protein processing plant, a corn oil processing plant, an ethanol and protein processing plant, an ethanol and corn oil processing plant, or an ethanol, corn oil and protein processing plant, and the fluid process stream may be a corn mash stream. The enzyme composition may be pulse dosed into the fluid stream at one or more points of the processing plant, such as before one or more process screens of a protein separation unit. For comparison, an equivalent dose of the enzyme composition is administered continuously at the one or more points. For example, pulse dosing to provide a concentration of 250 ppm, 500 ppm, 750 ppm, or 1000 ppm of the enzyme composition is performed for 60 seconds every 60 minutes, or 30 seconds every 30 minutes, and is compared to continuous dosing using an enzyme composition providing a concentration of 4.2 ppm, 8.3 ppm, 12.5 ppm, or 16.7 ppm, respectively. Evaluated parameters may include pressure, temperature, and/or flow rate over time. Pulse dosing to a given concentration can provide superior results as evidenced by smaller pressure increases, smaller temperature drops, smaller flow rate drops, and/or greater flow rates over time compared to only using a surfactant or surfactant-containing composition and/or using continuous dosing of such a composition.

### Example 4

Starch is baked onto one or more process screens to mimic deposits that occur on processing plant components as the processing plant is operated. The components with baked-on starch are immersed continuously or intermittently in a fluid (e.g., a corn mash) containing an enzyme composition alone and/or with a surfactant or a surfactant-containing composition. The components are immersed in fluids containing 250 ppm, 500 ppm, 750 ppm, or 1000 ppm of the enzyme composition for a period of time, with or without pulse dosing. The period of time may be 2 hours, 4 hours, 6 hours, 8 hours, 12 hours, 18 hours, or 24 hours. Components prepared the same way are immersed in fluids containing an equivalent dose of a surfactant or surfactant-containing composition, respectively, for the same period of time. Components also may be immersed in fluids containing no enzyme composition or no surfactant/surfactant-containing composition for the same period of time. The fluids may be maintained at a temperature similar to temperatures employed at the processing plant. When the period of time has elapsed, the components are evaluated. The amount of starch remaining on the components is evaluated (e.g., visually) and the ease of removing the starch is evaluated. Different cleaning methods, such as pressure washing, hydroblasting, cleaning with caustics, and/or other suitable methods, are evaluated for comparison. The enzyme composition can provide superior results as evidenced by easier cleaning of components subjected to the enzyme composition compared to components subjected to fluids with just a surfactant (and/or just a surfactant-containing composition) or in fluids with no cleaning formulation.

In view of the many possible embodiments to which the principles of the present disclosure may be applied, it should be recognized that the illustrated embodiments are only preferred examples and should not be taken as limiting the scope of the present disclosure. Rather, the scope is defined by the following claims. I therefore claim as my invention all that comes within the scope and spirit of these claims.

I claim:

1. A method for cleaning a process screen, comprising:
administering an enzyme composition comprising an amylase enzyme, a protease enzyme, a lipase enzyme, or any combination thereof to a process stream produced during biomass-based processing, wherein the process stream is a process stream that is exposed to fiber separation or protein separation; provided that the enzyme composition is not administered directly to a fermentation unit or a liquefaction unit of the biomass-based processing.

2. The method of claim 1, wherein administering comprises administering the enzyme composition from an enzyme composition containment unit to the process stream, wherein the enzyme composition containment unit is fluidly coupled to the process stream.

3. The method of claim 1, wherein administering comprises pulse dosing the enzyme composition into the process stream for a period of x seconds every y minutes with no administration of the enzyme composition between the periods of x seconds, wherein x and y independently range from 1 to 500.

4. The method of claim 3, wherein x and y independently are from 15 to 90.

5. The method of claim 3, wherein x=y=30.

6. The method of claim 3, wherein an amount of the enzyme composition is administered into the process stream to provide a concentration of 15 ppm to 1,500 ppm of the enzyme composition in the process stream during the period of x seconds.

7. The method of claim 1, wherein the amylase enzyme is an α-amylase, a β-amylase, a γ-amylase, or any combination thereof.

8. The method of claim 1, wherein the enzyme composition is a separate composition from any composition comprising an enzyme that is used to in any liquefaction, fermentation, and/or saccharification step of the biomass-based processing.

9. The method of claim 1, wherein the enzyme composition further comprises a surfactant.

10. The method of claim 1, wherein the method further comprises combining the enzyme composition with a surfactant-containing composition.

11. The method of claim 10, wherein combining the enzyme composition with the surfactant-containing composition comprises mixing the enzyme composition with the surfactant-containing composition prior to administering the enzyme composition into the process stream or separately, or sequentially or simultaneously adding the enzyme composition and the surfactant-containing composition to the process stream.

12. The method of claim 1, wherein the method further comprises administering the enzyme composition to one or more process screens used in a fiber separation unit of the biomass-based processing.

13. A method for cleaning a process screen, comprising:
administering an enzyme composition comprising an amylase enzyme, a protease enzyme, a lipase enzyme, or any combination thereof to a process stream produced during biomass-based processing, wherein (i) the process stream flows between a liquefaction unit and a fermentation unit used in the biomass-based processing; (ii) the process screen is located between the liquefaction unit and the fermentation unit; and (ii) the enzyme composition is administered upstream of the process screen; provided that the enzyme composition is not administered directly to the fermentation unit or the liquefaction unit of the biomass.

14. A method for cleaning a process screen, comprising:
administering an enzyme composition comprising an amylase enzyme to the process screen during biomass-based processing, wherein (i) the process screen is located between the liquefaction unit and the fermentation unit; and (ii) the enzyme composition is administered to the process screen by way of a cleaning stream that is separate from any process stream of the biomass-based processing.

15. The method of claim 14, wherein administering comprises administering the enzyme composition from an enzyme composition containment unit to the cleaning stream, wherein the enzyme composition containment unit is fluidly coupled to the cleaning stream.

16. The method of claim 14, wherein administering comprises pulse dosing the enzyme composition into the cleaning stream for a period of x seconds every y minutes with no administration of the enzyme composition between the periods of x seconds, wherein x and y independently range from 1 to 500.

17. The method of claim 16, wherein x and y independently are from 15 to 90.

18. The method of claim 16, wherein x=y=30.

19. The method of claim 16, wherein an amount of the enzyme composition is administered into the cleaning stream to provide a concentration of 15 ppm to 1,500 ppm of the enzyme composition in the cleaning stream during the period of x seconds.

20. The method of claim 14, wherein the amylase enzyme is an α-amylase, a β-amylase, a γ-amylase, or any combination thereof and/or the enzyme composition further comprises a surfactant.

21. The method of claim 14, wherein the method further comprises combining the enzyme composition with a surfactant-containing composition.
